(19) United States
(12) Patent Application Publication
(10) Pub. No.: US 2024/0026266 A1
(43) Pub. Date: Jan. 25, 2024

(54) CELL CULTURE BAG

(71) Applicant: NAN YA PLASTICS CORPORATION, Taipei (TW)

(72) Inventors: TE-CHAO LIAO, Taipei (TW); Min-Fan Chung, Taoyuan City (TW); CHING-YAO YUAN, Taipei (TW); CHIA-JUNG CHANG, Taipei (TW)

(21) Appl. No.: 18/063,634

(22) Filed: Dec. 8, 2022

(30) Foreign Application Priority Data
Jul. 12, 2022 (TW) 111126002

(51) Int. Cl. C12M 1/00

(52) U.S. Cl. CPC C12M 23/14; C12M 23/26; C12M 23/34; C12M 23/20

(57) ABSTRACT

A cell culture bag is provided. The cell culture bag includes a plastic bag body and a hydration layer. A culture space is formed in the plastic bag body. The plastic bag body has a zwitterionic polymer plastic surface facing the culture space. A material forming the zwitterionic polymer plastic surface includes a zwitterionic polymer. The hydration layer is attached onto the zwitterionic polymer plastic surface, and the culture space is surrounded by the hydration layer.

### CROSS-REFERENCE TO RELATED PATENT APPLICATION

This application claims the benefit of priority to Taiwan Patent Application No. 111126002, filed on Jul. 12, 2022. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a cell culture bag, and more particularly to a cell culture bag that has an excellent breathability.

### BACKGROUND OF THE DISCLOSURE

During cell therapy, cells harvested from patients are cultured in a cell culture bag. After adequate proliferation, cultured cells are taken out and then injected into the human body.

Oxygen is needed during a cell growth period, and carbon dioxide is discharged. Therefore, in order to provide a suitable growth environment, the most common way is to place the cell culture bag in an incubator. By adjusting gas environment conditions in the incubator, the atmosphere in the cell culture bag can also be controlled.

Accordingly, breathability of the cell culture bag becomes an important property, such that oxygen and carbon dioxide can smoothly permeate the cell culture bag for an inward diffusion or an outward diffusion to reach gas equilibrium.

Substances such as growth factors and proteins are released during the cell growth period. Over long-term usage, a protein layer may be formed by the attachment of growth factors and proteins on an inner surface of the cell culture bag. The breathability of the cell culture bag is negatively affected by the protein layer. Specifically, an oxygen transmission rate of the protein layer is 0.41 (cm³·mm)/(m²·atm), which is lower than that of a propylene membrane of 0.669(cm³·mm)/(m²·atm).

Therefore, how to improve the breathability of the cell culture bag by adjusting the material and the structure of the cell culture bag to overcome the aforesaid drawbacks has become one of the important issues to be addressed in the related field.

### SUMMARY OF THE DISCLOSURE

In response to the above-referenced technical inadequacy, the present disclosure provides a cell culture bag.

In one aspect, the present disclosure provides a cell culture bag. The cell culture bag includes a plastic bag body and a hydration layer. The plastic bag body has a culture space formed in the plastic bag body and a zwitterionic polymer plastic surface facing the culture space. A material for forming the zwitterionic polymer plastic surface includes a zwitterionic polymer. The hydration layer is attached onto the zwitterionic polymer plastic surface. The culture space is surrounded by the hydration layer.

In certain embodiments, one molecular end of the zwitterionic polymer has an acrylate group, and another molecular end of the zwitterionic polymer has an anionic moiety or a cationic moiety.

In certain embodiments, an anionic moiety of the zwitterionic polymer is selected from the group consisting of: a phosphate group, a sulfonic acid group, and a carboxylic acid group, and a cationic moiety of the zwitterionic polymer is a quaternary ammonium group.

In certain embodiments, the zwitterionic polymer includes 2-methacryloyloxyethylpho sphoric choline, sulfobetaine methacrylate, carboxybetaine methyl methacrylate, or any combination thereof.

In certain embodiments, a number average molecular weight of the zwitterionic polymer ranges from 5000 g/mol to 10000 g/mol.

In certain embodiments, the plastic bag body and the zwitterionic polymer plastic surface are integrally formed.

In certain embodiments, the plastic bag body and the zwitterionic polymer plastic surface are formed from a composite polymer material. Based on a total weight of the composite polymer material being 100 wt %, an amount of the zwitterionic polymer ranges from 1 wt % to 3 wt %.

In certain embodiments, the zwitterionic polymer plastic surface is formed on the plastic bag body by coating.

In certain embodiments, based on a total weight of the plastic bag body being 100 wt %, an amount of the zwitterionic polymer ranges from 50 ppm to 1000 ppm.

In certain embodiments, a thickness of the plastic bag body ranges from 50 μm to 400 μm.

In certain embodiments, a water vapor transmission rate of the cell culture bag ranges from 2 g/(m²·day) to 10 g/(m²·day), and an oxygen transmission rate of the cell culture bag is higher than 200 g/(m²·day).

Therefore, in the cell culture bag provided by the present disclosure, by virtue of “the plastic bag body having a zwitterionic polymer plastic surface” and “the culture space being surrounded by the hydration layer,” the probability of formation of the protein layer can be reduced such as to maintain the breathability of the cell culture bag against degradation.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic side view of a cell culture bag of the present disclosure; and

FIG. 2 is a schematic cross-sectional view taken along line II-II of FIG. 1.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

The present disclosure provides a cell culture bag that has an excellent breathability (toward water vapor and oxygen). Therefore, when the cell culture bag is placed in the incubator, gases can permeate the cell culture bag so as to maintain an atmosphere in the cell culture bag.

In addition, proteins are difficult to attach onto the cell culture bag of the present disclosure. Growth factors and proteins are released during cell growth period. When growth factors and proteins are attached on the cell culture bag, a protein layer is formed on an inner surface of the cell culture bag, so that the breathability of the cell culture bag is negatively affected by a formation of the protein layer.

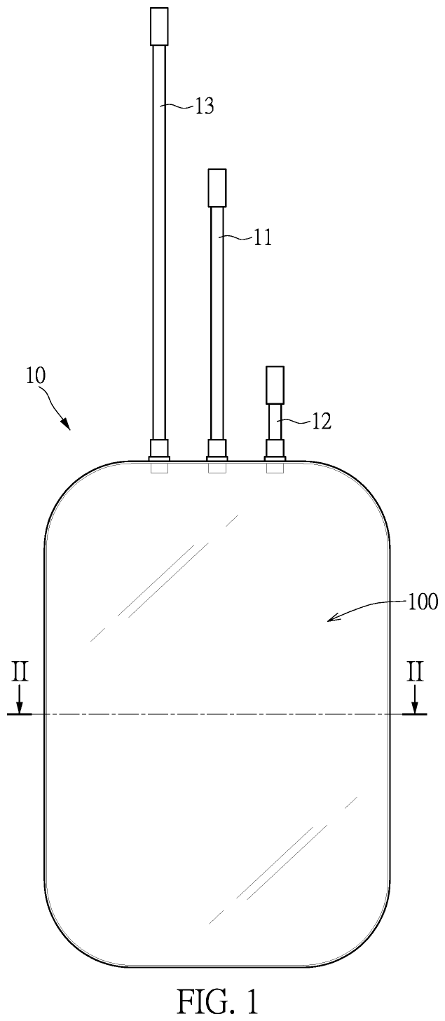

Referring to FIG. 1, the cell culture bag of the present disclosure is formed from a plastic bag body 10 acting as a main body. A culture space 100 is formed in the plastic bag body 10. The plastic bag body 10 is a transparent bag body (light transmittance above 90%) in order to observe the state of cells.

The plastic bag body 10 should have a high oxygen transmission rate and a high water vapor transmission rate, so as to maintain the atmosphere of the culture space 100. In an exemplary embodiment, a material for forming the plastic bag body 10 is an ethylene-vinyl acetate copolymer (EVA) or a low density polyethylene (LDPE). Preferably, a thickness of the plastic bag body 10 ranges from 50 μm to 150 μm.

For the convenience of use, a medium injection tube 11, a cell injection tube 12, and a sampling tube 13 are mounted on the plastic bag body 10.

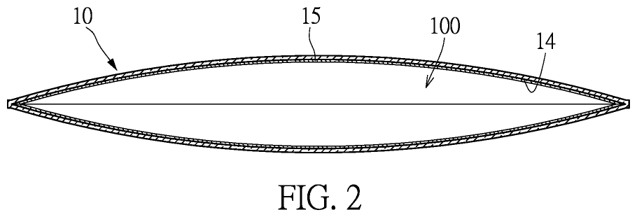

Referring to FIG. 1 and FIG. 2, the zwitterionic polymer plastic surface 14 can prevent growth factors and proteins released from cells from attaching onto the plastic bag body 10 and forming a protein layer. The zwitterionic polymer plastic surface 14 faces the culture space 100.

An anionic moiety and a cationic moiety are exposed from the zwitterionic polymer plastic surface 14. Water vapor in the air can combine with the anionic moiety and the cationic moiety. Therefore, a hydration layer 15 is formed by attaching on the zwitterionic polymer plastic surface 14, such that the culture space 100 is surrounded by the hydration layer 15.

After being released from cells, the growth factors and the proteins contact the hydration layer 15. An adhesion of the growth factors and the proteins toward the hydration layer 15 is weaker than an adhesion of the growth factors and the proteins toward the plastic bag body 10. Therefore, the hydration layer 15 can prevent the formation of the protein layer.

In other words, the dispositions of the zwitterionic polymer plastic surface 14 and the hydration layer 15 can prevent the formation of the protein layer. Accordingly, the cell culture bag of the present disclosure can maintain its original breathability during the cell culture process.

Specifically, a material for forming the zwitterionic polymer plastic surface 14 includes a zwitterionic polymer. Based on a total area of the zwitterionic polymer plastic surface 14 being 100%, an amount of the zwitterionic polymer ranges from 0.1 wt % to 3 wt %.

In an exemplary embodiment, the anionic moiety of the zwitterionic polymer is selected from the group consisting of: a phosphate group, a sulfonic acid group, and a carboxylic acid group. A cationic moiety of the zwitterionic polymer is a quaternary ammonium group. A number average molecular weight of the zwitterionic polymer ranges from 5000 g/mol to 10000 g/mol, so as to avoid the attachment of the proteins. However, the present disclosure is not limited thereto.

In an exemplary embodiment, one molecular end of the zwitterionic polymer has an acrylate group, another molecular end of the zwitterionic polymer has an anionic moiety (or a cationic moiety), and a cationic moiety (anionic moiety) is located between the acrylate group and the anionic moiety (or a cationic moiety).

In addition, a distance between a center atom of the anionic moiety and a center atom of the cationic moiety ranges from 1 carbon atom to 20 carbon atoms. Preferably, the distance between a center atom of the anionic moiety and a center atom of the cationic moiety ranges from 1 carbon atom to 5 carbon atoms. Accordingly, the hydration layer 15 can be easily formed on the zwitterionic polymer plastic surface 14.

In a preferable embodiment, the zwitterionic polymer can be 2-methacryloyloxyethyl phosphorylcholine (MPC), sulfobetaine methacrylate (SBMA), carboxybetaine methacrylate (CBMA), or any combination thereof.

Specific structural formulas of 2-methacryloyloxyethyl phosphorylcholine (MPC), sulfobetaine methacrylate (SBMA), carboxybetaine methacrylate (CBMA) are shown as below. “m” and “n” are independently an integer between 1 and 20.

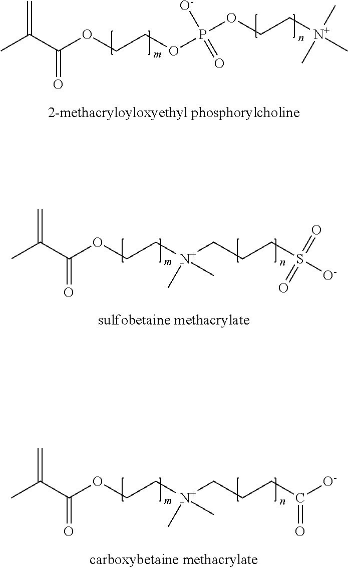

It should be noted that one molecular end of 2-methacryloyloxyethyl phosphorylcholine, sulfobetaine methacrylate, and carboxybetaine methacrylate is an acrylate group. Another molecular end of 2-methacryloyloxyethyl phosphorylcholine is a cationic moiety (quaternary ammonium group). Another molecular end of sulfobetaine methacrylate and another molecular end of carboxybetaine methacrylate are an anionic moiety (sulfonic acid group or carboxylic acid group). Further, a distance, in carbon atoms, between a center atom of the anionic moiety and a center atom of the cationic moiety in 2-methacryloyloxyethyl phosphorylcholine, sulfobetaine methacrylate, and carboxybetaine methacrylate is 3 carbon atoms.

### First Embodiment

The zwitterionic polymer plastic surface and the plastic bag body are integrally formed in a first embodiment of the present disclosure.

Specifically, the plastic bag body is formed from a composite polymer material. The composite polymer material includes a main material and the zwitterionic polymer. The main material and the zwitterionic polymer are melted and mixed so as to form the composite polymer material.

The composite polymer material contains the zwitterionic polymer, and the plastic bag body is formed from the composite polymer material. Therefore, the plastic bag body has the zwitterionic polymer plastic surface from which the anionic moiety and the cationic moiety are exposed. Moreover, an amount ratio of the zwitterionic polymer on the zwitterionic polymer plastic surface is approximately the same as an amount ratio of the zwitterionic polymer in the composite polymer material.

Specifically, based on a total weight of the composite polymer material being 100 wt %, an amount of the zwitterionic polymer ranges from 1 wt % to 3 wt %.

In an exemplary embodiment, ethylene-vinyl acetate copolymer particles or low density polyethylene particles are used as a main material of the plastic bag body. The ethylene-vinyl acetate copolymer particles or the low density polyethylene particles are melted and mixed with the zwitterionic polymer, so as to manufacture the plastic bag body that has the zwitterionic polymer plastic surface.

### Second Embodiment

The zwitterionic polymer plastic surface is formed on the plastic bag body by a coating process in a second embodiment of the present disclosure.

Specifically, the ethylene-vinyl acetate copolymer particles or the low density polyethylene particles are melted to form an EVA membrane or a LDPE membrane. Subsequently, a coating solution that contains the zwitterionic polymer is coated onto the EVA membrane or the LDPE membrane. After drying, the zwitterionic polymer plastic surface is formed onto the EVA membrane or the LDPE membrane. Finally, the EVA membrane or the LDPE membrane that has the zwitterionic polymer plastic surface can be used to manufacture the cell culture bag of the present disclosure.

In an exemplary embodiment, zwitterionic polymer powder is dissolved in alcohol, such as methanol or ethanol, so as to prepare the coating solution. Specifically, a concentration of the zwitterionic polymer in the coating solution ranges from 0.05 wt % to 0.2 wt %. After being coated, based on a total weight of the plastic bag body being 100 wt %, an amount of the zwitterionic polymer ranges from 50 ppm to 1000 ppm.

In order to strengthen an adhesion between the zwitterionic polymer plastic surface and the EVA membrane or the LDPE membrane, the EVA membrane or the LDPE membrane can be treated by a hydrophobic modification before coating the coating solution.

The cell culture bags of the first embodiment and the second embodiment both have the zwitterionic polymer plastic surface facing the culture space in order to form a hydration layer. Accordingly, the protein layer is difficult to be formed on the inner surface of the cell culture bag.

### EXPERIMENTAL DATA

In order to prove that the cell culture bag of the present disclosure has an excellent breathability, the cell culture bag in each of Examples 1 to 3 and Comparative Example 1 is manufactured according to steps as follows. The cell culture bag in each of Examples 1 to 3 and Comparative Example 1 undergoes a water vapor permeability rate test and an oxygen permeability rate test.

### Examples 1 to 3

The LDPE particles are used as the main material. The main material and the zwitterionic polymer powder are melted and mixed to form the composite polymer material. The difference between Examples 1 to 3 is that different zwitterionic polymer powders are used. Specific components and contents of the composite polymer material are listed in Table 1.

The composite polymer material is used to manufacture the cell culture bag having a thickness of 100 μm. According to the ASTM F1249 test method, the water vapor permeability rate of the cell culture bag is measured. According to the ASTM D3985 test method, the oxygen permeability rate of the cell culture bag is measured. The results of the water vapor permeability rate and the oxygen permeability rate of the cell culture bag are listed in Table 1. In addition, after a 7-day culture period, the water vapor permeability rate and the oxygen permeability rate of the cell culture bag are measured and also listed in Table 1.

### Comparative Example 1

The cell culture bag of Comparative Example 1 is manufactured by a method similar to that by which the cell culture bag of Example 1 is manufactured. The difference is that the composite polymer material in Comparative Example 1 does not contain the zwitterionic polymer. Specific components and contents of the composite polymer material are listed in Table 1.

The composite polymer material is used to manufacture the cell culture bag having a thickness of 100 μm. According to the ASTM F1249 test method, the water vapor permeability rate of the cell culture bag is measured. According to the ASTM D3985 test method, the oxygen permeability rate of the cell culture bag is measured. The results of the water vapor permeability rate and the oxygen permeability rate of the cell culture bag are listed in Table 1.

In addition, after the 7-day culture period, the water vapor permeability rate and the oxygen permeability rate of the cell culture bag are measured and also listed in Table 1.

TABLE 1

| | | Comparative | | | |
|---|---|---|---|---|---|
| | | Example 1 | Example 1 | Example 2 | Example 3 |
| Composite polymer | | LDPE | LDPE + | LDPE + | LDPE + |
| material | | | 1 wt % | 1 wt % | 1 wt % |
| | | | MPC | SBMA | CBMA |
| Water vapor | Before | 4.03 | 7.18 | 5.18 | 3.15 |
| permeability | culture | g/m²/day | g/m²/day | g/m²/day | g/m²/day |
| | After | 0.51 | 6.05 | 4.59 | 2.88 |
| | culture | g/m²/day | g/m²/day | g/m²/day | g/m²/day |
| Decreasing rate | | 87.3% | 15.7% | 11.4% | 8.6% |
| of water vapor | | | | | |
| permeability | | | | | |
| Oxygen | Before | >200 | >200 | >200 | >200 |
| permeability | culture | g/m²/day | g/m²/day | g/m²/day | g/m²/day |
| | After | 158 | 158 | 158 | 158 |
| | culture | g/m²/day | g/m²/day | g/m²/day | g/m²/day |

According to Table 1, the cell culture bag has excellent water vapor permeability. After the 7-day culture period, the water vapor permeability of the cell culture bag can still be higher than 2.5 g/m²/day. Preferably, the water vapor permeability of the cell culture bag can still be higher than 2 6 g/m² /day. More preferably, the water vapor permeability of the cell culture bag can still be higher than 2 8 g/m²/day. After the 7-day culture period, a decreasing rate of the water vapor permeability of the cell culture bag ranges from 5% to 20%. Preferably, the decreasing rate of the water vapor permeability of the cell culture bag ranges from 7% to 16%.

### Beneficial Effects of the Embodiments

In conclusion, in the cell culture bag provided by the present disclosure, by virtue of “the plastic bag body having a zwitterionic polymer plastic surface” and “the culture space being surrounded by the hydration layer,” the probability of formation of the protein layer can be reduced so as to maintain the breathability of the cell culture bag against degradation.

Further, by virtue of “one molecular end of the zwitterionic polymer having an acrylate group, and another molecular end of the zwitterionic polymer having an anionic moiety or a cationic moiety,” proteins can be prevented from attaching onto the cell culture bag of the present disclosure.

Further, by virtue of “the plastic bag body and the zwitterionic polymer plastic surface being integrally formed, and based on a total weight of the composite polymer material being 100 wt %, an amount of the zwitterionic polymer ranging from 1 wt % to 3 wt %,” proteins can be prevented from attaching onto the cell culture bag of the present disclosure.

Further, by virtue of “the zwitterionic polymer plastic surface being formed on the plastic bag body by coating” and “based on a total weight of the plastic bag body being 100 wt %, an amount of the zwitterionic polymer ranging from 50 ppm to 1000 ppm,” proteins can be prevented from attaching onto the cell culture bag of the present disclosure.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A cell culture bag, comprising:
a plastic bag body having a culture space formed in the plastic bag body and a zwitterionic polymer plastic surface facing the culture space; wherein a material for forming the zwitterionic polymer plastic surface includes a zwitterionic polymer; and
a hydration layer attached onto the zwitterionic polymer plastic surface, the culture space being surrounded by the hydration layer.

2. The cell culture bag according to claim 1, wherein one molecular end of the zwitterionic polymer has an acrylate group, and another molecular end of the zwitterionic polymer has an anionic moiety or a cationic moiety.

3. The cell culture bag according to claim 1, wherein an anionic moiety of the zwitterionic polymer is selected from the group consisting of: a phosphate group, a sulfonic acid group, and a carboxylic acid group, and a cationic moiety of the zwitterionic polymer is a quaternary ammonium group.

4. The cell culture bag according to claim 1, wherein the zwitterionic polymer includes 2-methacryloyloxyethylphosphoric choline, sulfobetaine methacrylate, carboxybetaine methyl methacrylate, or any combination thereof.

5. The cell culture bag according to claim 1, wherein a number average molecular weight of the zwitterionic polymer ranges from 5000 g/mol to 10000 g/mol.

6. The cell culture bag according to claim 1, wherein the plastic bag body and the zwitterionic polymer plastic surface are integrally formed.

7. The cell culture bag according to claim 6, wherein the plastic bag body and the zwitterionic polymer plastic surface are formed from a composite polymer material, and based on a total weight of the composite polymer material being 100 wt %, an amount of the zwitterionic polymer ranges from 1 wt % to 3 wt %.

8. The cell culture bag according to claim 1, wherein the zwitterionic polymer plastic surface is formed on the plastic bag body by coating.

9. The cell culture bag according to claim 8, wherein, based on a total weight of the plastic bag body being 100 wt %, an amount of the zwitterionic polymer ranges from 50 ppm to 1000 ppm.

10. The cell culture bag according to claim 1, wherein a thickness of the plastic bag body ranges from 50 μm to 400 μm.

11. The cell culture bag according to claim 1, wherein a water vapor transmission rate of the cell culture bag ranges from 2 g/(m²·day) to 10 g/(m²·day), and an oxygen transmission rate of the cell culture bag is higher than 200 g/(m²·day).
